(19) United States
(12) Patent Application Publication
(10) Pub. No.: US 2026/0178928 A1
(43) Pub. Date: Jun. 25, 2026

(54) ACTIVE LEARNING TECHNIQUES FOR DEVELOPING MAP PREDICTION MACHINE LEARNING MODELS TO GENERATE MAPS OF BIOLOGY

(71) Applicant: Recursion Pharmaceuticals, Inc., Salt Lake City, UT (US)

(72) Inventors: Ihor NEPOROZHNII, Toronto (CA); Jason Siyanda HARTFORD, London (GB); Julien ROY, Montreal (CA); Oscar MENDEZ LUCIO, Madrid (ES); Peter Foster MCCLEAN, Centerville, UT (US)

(21) Appl. No.: 18/990,261

(22) Filed: Dec. 20, 2024

(51) Int. Cl. G06N 3/096; G16B 40/00; G16C 20/70

(52) U.S. Cl. CPC G06N 3/096; G16B 40/00; G16C 20/70

(57) ABSTRACT

The present disclosure relates to systems, non-transitory computer-readable media, and methods for efficiently acquiring biological data via active machine learning. In particular, in some embodiments, the disclosed systems generate, utilizing a map prediction machine learning model, similarity prediction confidence scores for a plurality of perturbation pairs. In addition, in some embodiments, the disclosed systems determine, from the similarity prediction confidence scores utilizing an acquisition function, a perturbation pair for developing a ground truth similarity. Moreover, in some embodiments, the disclosed systems generate a tuned map prediction model by comparing a similarity prediction for the perturbation pair generated by the map prediction machine learning model with the ground truth similarity. Furthermore, in some embodiments, the disclosed systems utilize, in response to determining that a measure of confidence for the tuned map prediction model satisfies a stopping criterion, the tuned map prediction model to generate a blended map of biology.

### BACKGROUND

Recent years have seen developments in hardware and software platforms for training and utilizing machine learning models for generating predictions. For example, existing systems utilize large volumes of training data to teach machine learning models to generate intelligent predictions corresponding to complex biological interactions between genes, compounds, and/or proteins. Despite these recent developments, existing systems suffer from a number of technical deficiencies, particularly with regard to accuracy, efficiency, and operational flexibility in implementing machine learning technologies.

### BRIEF SUMMARY

Embodiments of the present disclosure provide benefits and/or solve one or more problems in the art with systems, non-transitory computer-readable media, and methods for efficient biological data acquisition through active machine learning. In some embodiments, the disclosed systems generate blended maps of biology comprising both observed data and inference data of relationships between cell perturbations. To illustrate, in some embodiments, the disclosed systems utilize observed data to tune a map prediction machine learning model to accurately generate predicted relationships between various cell perturbations, such as compound perturbations and gene perturbations. For example, in some implementations, the disclosed systems utilize the map prediction model to determine confidence scores for the predicted relationships, and utilize an acquisition function to select perturbation pairs for testing based on the confidence scores. Moreover, in some embodiments, the disclosed systems develop ground truth similarities for the tested perturbation pairs to tune the map prediction model. With the tuned map prediction model, in some implementations, the disclosed systems generate similarity predictions for unobserved perturbation pairs to populate a map of biology, thereby reducing the number of experiments to run and enhancing efficiency of biological map acquisition computing systems.

The following description sets forth additional features and advantages of one or more embodiments of the disclosed methods, non-transitory computer-readable media, and systems. In some cases, such features and advantages are evident to a skilled artisan having the benefit of this disclosure, or may be learned by the practice of the disclosed embodiments.

### BRIEF DESCRIPTION OF THE DRAWINGS

The detailed description provides one or more embodiments with additional specificity and detail through the use of the accompanying drawings, as briefly described below.

FIG. 1 illustrates an overview of a map acquisition system in accordance with one or more embodiments.

FIG. 2 illustrates the map acquisition system utilizing active learning to tune a map prediction model for active map acquisition in accordance with one or more embodiments.

FIG. 3 illustrates the map acquisition system tuning a map prediction model to generate similarity predictions for cell perturbation pairs in accordance with one or more embodiments.

FIG. 4 illustrates the map acquisition system generating a plurality of maps of biology based on various inputs to a map prediction model in accordance with one or more embodiments.

FIG. 5 illustrates experimental results for the map acquisition system in accordance with one or more embodiments.

FIG. 6 illustrates a diagram of an environment in which a map acquisition system operates in accordance with one or more embodiments.

FIG. 7 illustrates a flowchart of a series of acts for generating maps of biology via active map acquisition in accordance with one or more embodiments.

FIG. 8 illustrates a block diagram of an example computing device for implementing one or more embodiments of the present disclosure.

### DETAILED DESCRIPTION

This disclosure describes one or more embodiments of a map acquisition system that utilizes active learning to tune a map prediction model to generate maps of biology comprising relationship data for cell perturbations. To illustrate, in some embodiments, the map acquisition system utilizes observed cell perturbation data to tune a map prediction machine learning model to accurately generate predicted relationships between various cell perturbations, such as compound perturbations and gene perturbations. In some embodiments, the map acquisition system utilizes the map prediction machine learning model to determine confidence scores for the predicted relationships. Additionally, in some embodiments, the map acquisition system utilizes the confidence scores and an acquisition function to select perturbation pairs for testing. Moreover, in some embodiments, the map acquisition system develops ground truth similarities for the perturbation pairs and uses the ground truth similarities to tune the map prediction machine learning model. With the tuned map prediction machine learning model, the map acquisition system generates similarity predictions for unobserved perturbation pairs to populate a blended map of biology comprising both observed data and inference data.

In pharmaceutical compound discovery, highly automated high-throughput laboratories are used in conjunction with implementing computing systems to screen a large number of compounds in search of effective compounds. While these automated system experiments can be time consuming and computationally expensive, the map acquisition system can enhance efficiency by determining a subset of compounds to explore and predicting the biological effects of the remaining experiments. In some embodiments, the map acquisition system selects a small set of candidates to achieve a desired level of accuracy for the system as a whole. In some cases, there is heterogeneity in the difficulty of the prediction problem across the input space, and the map acquisition system selectively obtains labels for the most difficult examples in the acquisition pool, thereby leaving less difficult examples to remain in the inference set. Using such inference set design, the map acquisition system leads to better overall system performance.

To further illustrate, in some implementations, the map acquisition system uses an uncertainty-based active learning solution to prune out the more challenging examples. For example, the map acquisition system uses an explicit stopping criterion that stops running training and experiment feedback loops when it has sufficient confidence that the system has reached the target performance. Moreover, empirical studies on a real-world large-scale biological assay show that, by deploying active learning for inference set design, the map acquisition system provides significant reduction in experimental cost while obtaining high system performance.

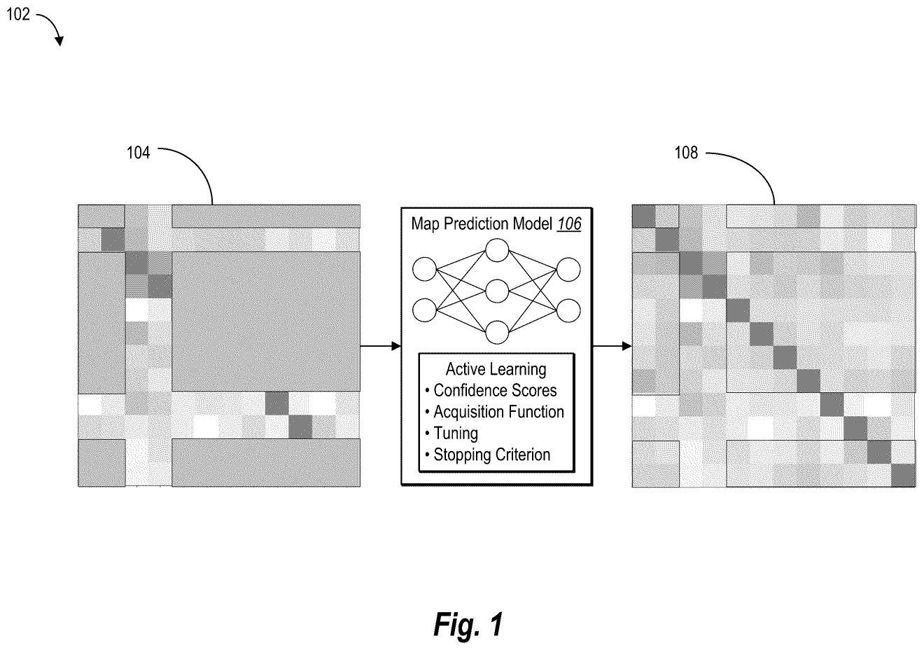

As just mentioned, in some embodiments, the map acquisition system 102 utilizes active learning to tune a map prediction machine learning model to generate maps of biology. For instance, FIG. 1 illustrates the map acquisition system 102 utilizing active learning with a map prediction model to generate a blended map of biology in accordance with one or more embodiments.

Specifically, FIG. 1 shows the map acquisition system 102 generating an initial map of biology 104. In some cases, the initial map of biology 104 has similarity data (e.g., observation data on cell perturbation similarities) for some cell perturbation pairs, but is incomplete (e.g., missing similarity data) as to other cell perturbation pairs. Moreover, in some embodiments, the map acquisition system 102 uses a map prediction machine learning model 106 (or map prediction model 106) to populate the initial map of biology 104 with additional similarity data (e.g., inference data for cell perturbation similarities) to generate a blended map of biology 108.

To illustrate, in some implementations, the map acquisition system 102 uses active learning to develop observed ground truth data to tune the map prediction model 106 to generate inference data. For instance, the map acquisition system 102 generates confidence scores for similarity predictions that indicate a measure of prediction difficulty for the perturbation pairs. Moreover, in some embodiments, the map acquisition system 102 employs an acquisition function to determine one or more perturbation pairs to test to develop a ground truth similarity. In addition, in some implementations, the map acquisition system 102 uses a stopping criterion to determine that the map prediction model 106 has attained an acceptable level of accuracy. Furthermore, the map acquisition system 102 generates similarity predictions for unobserved perturbation pairs using the map prediction model 106. The map acquisition system 102 populates (e.g., adds similarity data to) the initial map of biology 104 to generate the blended map of biology 108.

A cell perturbation includes a modification, or treatment applied to a biological cell, such as by a chemical compound perturbation (e.g., a drug treatment) or a gene knockout perturbation. A perturbation pair includes a match of two cell perturbations for comparing their biological relationships (e.g., effects on a cell). For example, a perturbation pair includes a gene knockout perturbation paired with (e.g., compared with) a chemical compound perturbation. As another example, a perturbation pair includes a gene knockout perturbation paired with (e.g., compared with) another gene knockout perturbation. As yet another example, a perturbation pair includes a chemical compound perturbation paired with (e.g., compared with) another chemical compound perturbation. A perturbation can include a gene, small molecule (e.g., therapeutic compound or drug), biologic, or other treatment.

A similarity prediction includes a score or a classification that denotes a biological similarity between perturbations. For example, a similarity prediction denotes a predicted similarity of the bioactivity of a chemical compound (as applied to a living cell) to the bioactivity of a gene perturbation (as applied to the living cell). For instance, a similarity prediction can indicate a similarity score or classification for a level of similarity in cell development, impact, or expression between a compound and one or more other perturbations.

In some embodiments, a similarity prediction is a phenomic similarity prediction. To illustrate, a phenomic similarity prediction includes a prediction of how similarly two cell perturbations will affect a phenotypic expression of a cell. Alternatively, in some embodiments, a similarity prediction is a transcriptomic similarity prediction. For instance, a transcriptomic similarity prediction includes a prediction of how similarly two cell perturbations will affect a transcriptome of a cell (e.g., RNA molecules present in the cell).

A map prediction machine learning model (or map prediction model) includes a machine learning model designed to generate similarity predictions for biological cell perturbations. In some embodiments, a map prediction model also generates confidence scores for the similarity predictions that indicate a degree of certainty of how accurate the similarity predictions are. In some embodiments, the map acquisition system 102 utilizes a map prediction model as described in U.S. patent application Ser. No. 18/753,906, filed on Jun. 25, 2024, entitled DETERMINING PHENOMIC RELATIONSHIPS BETWEEN COMPOUNDS AND CELL PERTURBATIONS UTILIZING MACHINE LEARNING MODELS, the contents of which are incorporated by reference herein in their entirety.

A machine learning model includes a computer representation that is tunable (e.g., trained) based on inputs to approximate unknown functions used for generating corresponding outputs. In particular, in one or more embodiments, a machine learning model is a computer-implemented model that utilizes algorithms to learn from, and make predictions on, known data by analyzing the known data to learn to generate outputs that reflect patterns and attributes of the known data. For instance, in some cases, a machine learning model includes, but is not limited to, a neural network (e.g., a convolutional neural network, recurrent neural network, or other deep learning network), a decision tree (e.g., a gradient boosted decision tree), support vector learning, Bayesian networks, a transformer-based model, a diffusion model, or a combination thereof.

Similarly, a neural network includes a machine learning model that is trainable and/or tunable based on inputs to determine classifications and/or scores, or to approximate unknown functions. For example, in some cases, a neural network includes a model of interconnected artificial neurons (e.g., organized in layers) that communicate and learn to approximate complex functions and generate outputs based on inputs provided to the neural network. In some cases, a neural network refers to an algorithm (or set of algorithms) that implements deep learning techniques to model high-level abstractions in data. A neural network includes various layers such as an input layer, one or more hidden layers, and an output layer that each perform tasks for processing data. For example, a neural network includes a deep neural network, a convolutional neural network, a diffusion neural network, a recurrent neural network (e.g., an LSTM), a graph neural network, a transformer, or a generative adversarial neural network.

In some embodiments, the map acquisition system 102 utilizes a transformer-based model as the map prediction model 106 that analyzes a graph representation of an input compound. For example, in some embodiments, the map acquisition system 102 utilizes one or more of the models described by Méndez-Lucio et al. in MolE: A Molecular Foundation Model for Drug Discovery, arXiv.2211.02657, November 2022, which is incorporated by reference herein in its entirety.

In some implementations, the map acquisition system 102 utilizes a multi-task model (e.g., a model with multiple task heads) as the map prediction model 106, whereby the map acquisition system 102 can generate similarity predictions for a plurality of (e.g., numerous) gene perturbations or other perturbations for a chemical compound. To illustrate, the map acquisition system 102 can train classification task heads (e.g., neural network prediction heads) for different gene perturbations. The map acquisition system 102 can then utilize the task heads to generate predictions for the gene perturbations from a structural feature representation of an input compound. For instance, the map acquisition system 102 can utilize a compound structure feature representation for the chemical compound to generate similarity predictions for genes in a library of thousands of genes.

As mentioned, conventional systems have a number of technical problems with regard to efficiency and accuracy of implementing computing devices. For example, existing systems are inefficient because they often require excessive experimental evaluations to evaluate biological similarities for cell perturbations, leading to great computational expense. For example, by running excessive experiments, existing systems cause undue burdens on memory and storage use, bandwidth capacity, and computational operations (e.g., generating machine learning embeddings).

In addition, existing systems are often inaccurate. For example, because existing systems often require a brute-force approach to biological data acquisition, they often are incomplete (e.g., missing similarity data). Furthermore, existing systems often generate inaccurate predicted data, as their training is performed via a random agent.

The map acquisition system 102 provides a variety of technical advantages relative to existing systems. For example, the map acquisition system 102 improves efficiency relative to conventional systems. Indeed, the map acquisition system 102 can reduce the number of experiments to run on cell perturbations, thereby enhancing efficiency of biological map acquisition. Accordingly, upon training a map prediction model, the map acquisition system 102 can utilize the map prediction model to generate similarity predictions (e.g., to identify genes or compounds that effect similar biological responses in cellular bioactivity) while avoiding much of the time and resources needed by conventional systems to execute robotic assays, generate and store machine learning embeddings, and compare such embeddings. Furthermore, by reducing the number of experimental assays to run, the map acquisition system 102 reduces computing time, processing power, bandwidth consumed, and memory required to analyze novel compounds, generate machine learning embeddings and similarity predictions, and transmit results over a computing network. By focusing on enhancing performance on a target set of samples and deciding which examples to label and which to predict, the map acquisition system provides a highly efficient tool for biological data acquisition.

Moreover, the map acquisition system 102 can provide enhanced accuracy of biosimilarity predictions by utilizing an active agent performing inference set design to train the map prediction model. In particular, the map acquisition system 102 improves overall system performance, even while reducing experimental costs. For example, the map acquisition system 102 provides substantial improvement in prediction accuracy by combining a confidence-based active learning acquisition function with an appropriate stopping criterion. In particular, the stopping criterion terminates a search for new molecules when a lower bound of target accuracy is exceeded, thereby providing inference data on unobserved perturbations that satisfies the accuracy threshold. Thus, by selecting the most challenging examples for testing and labeling, and leaving the easier examples for prediction, the map acquisition system demonstrates consistent and significant improvements across chemical datasets and real-world biological applications.

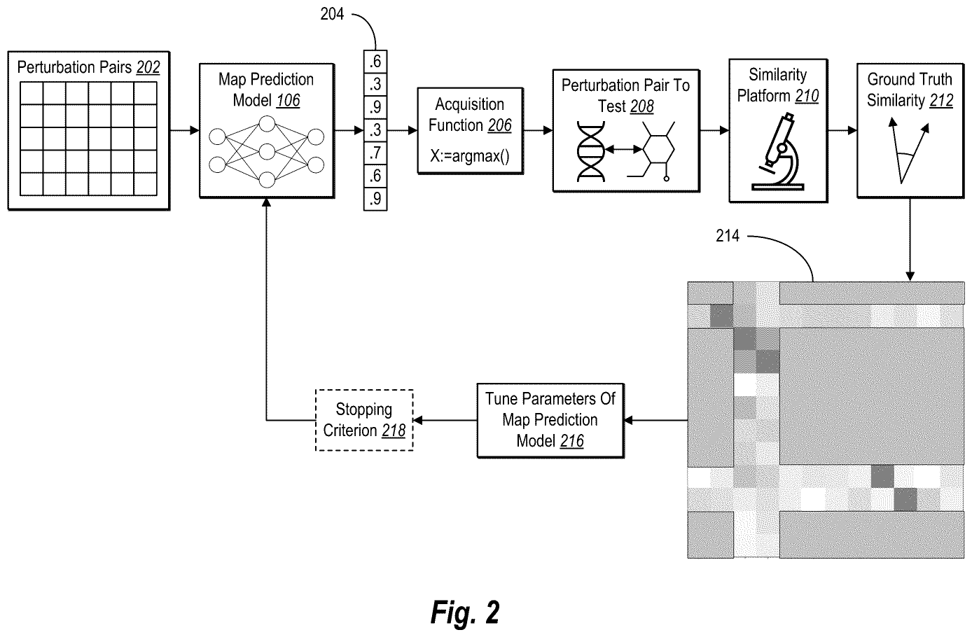

As mentioned, in some embodiments, the map acquisition system 102 tunes a map prediction model to generate blended maps of biology. For instance, FIG. 2 illustrates the map acquisition system 102 utilizing active learning to tune a map prediction model in accordance with one or more embodiments.

Specifically, FIG. 2 shows the map acquisition system 102 obtaining a plurality of perturbation pairs 202 to process through the map prediction model 106. For example, the map acquisition system 102 accesses a library of cell perturbations and generates a matrix of perturbation pairs for the library of cell perturbations. Additionally, FIG. 2 shows the map acquisition system 102 utilizing the map prediction model 106 to generate similarity prediction confidence scores 204 for the plurality of perturbation pairs 202. To illustrate, the map acquisition system 102 uses the map prediction model 106 to determine a level of confidence that represents, for a perturbation pair, a likelihood that the map prediction model 106 generates an accurate similarity prediction. For instance, a high similarity prediction confidence score represents that the corresponding similarity prediction for a particular perturbation pair has a high likelihood of being accurate.

For example, in some implementations, the map acquisition system 102 utilizes an intermediate layer of the map prediction model 106 to generate the confidence score. For example, the map acquisition system 102 strips a prediction layer (e.g., a softmax layer) for generating a similarity prediction and analyzes an intermediate layer that indicates a probability or likelihood of a particular prediction. The map acquisition system 102 can utilize the output of this intermediate layer as a confidence score.

In some embodiments, based on the similarity prediction confidence scores 204, the map acquisition system 102 utilizes an acquisition function 206 to determine a perturbation pair 208 to test. To illustrate, the map acquisition system 102 determines the perturbation pair 208 from the plurality of perturbation pairs 202 to test for developing a ground truth similarity. For example, in some embodiments, the map acquisition system 102 selects a perturbation pair with a low similarity prediction confidence score for testing. As another example, in some embodiments, the map acquisition system 102 selects a perturbation pair randomly for testing. In some implementations, the map acquisition system 102 utilizes the acquisition function 206 to determine that a confidence value for the perturbation pair 208 satisfies a testing criterion. Additional detail about the acquisition function 206 is given below.

Moreover, FIG. 2 shows the map acquisition system 102 using a similarity platform 210 to run an experiment for the perturbation pair 208 to develop a ground truth similarity 212. For example, in some implementations, the map acquisition system 102 accesses a first perturbation (e.g., a compound applied to a cell) to run a first experiment and a second perturbation (e.g., a gene knockout applied to a cell) to run a second experiment. Furthermore, in some implementations, the map acquisition system 102 compares the results of the experiments to determine the ground truth similarity 212.

To illustrate, in some implementations, the map acquisition system 102 utilizes a phenomic imaging platform as the similarity platform 210. For example, the map acquisition system 102 utilizes a camera to capture images (e.g., digital images) of biological cells exposed to chemical compounds or gene perturbations. Upon exposure to a compound or a gene knockout perturbation, a cell may undergo a change (e.g., a physical or biological change) expressed as a phenotypic change, which the map acquisition system 102 captures in the image of the cell.

Moreover, in some embodiments, the map acquisition system 102 generates embeddings for the results of the experiments, such as phenomic image embeddings. Thus, in some implementations, the map acquisition system 102 generates the ground truth similarity 212 by comparing a first embedding for the first perturbation with a second embedding for the second perturbation. For example, the map acquisition system 102 determines a cosine similarity (or feature space distance measure) between the first embedding and the second embedding.

To further illustrate, in some embodiments, the map acquisition system 102 performs (e.g., utilizing robotic assay implementation devices) cell perturbations and captures phenomic digital images of the perturbed cells. Specifically, the map acquisition system 102 performs a machine learning analysis on the digital images portraying the perturbed cells to generate embeddings from the phenomic digital images and compares the embeddings to identify inter-relationships between genes, proteins, compounds, and/or diseases. Thus, the map acquisition system 102 generates a phenomic similarity prediction from phenomic embeddings of a machine learning model generated from digital images portraying cells exposed to various perturbations.

To illustrate, in some implementations, the map acquisition system 102 generates phenomic embeddings as described in U.S. patent application Ser. No. 18/392,989, titled UTILIZING MACHINE LEARNING AND DIGITAL EMBEDDING PROCESSES TO GENERATE DIGITAL MAPS OF BIOLOGY AND USER INTERFACES FOR EVALUATING MAP EFFICACY, filed on Dec. 21, 2023 (hereinafter '989 Patent), which is incorporated by reference herein in its entirety. Additionally, in some cases, the map acquisition system 102 can utilize a machine learning model trained to generate predicted cell representations from masked cell representations as described in U.S. patent application Ser. No. 18/545,399, titled UTILIZING MASKED AUTOENCODER GENERATIVE MODELS TO EXTRACT MICROSCOPY REPRESENTATION AUTOENCODER EMBEDDINGS, filed on Dec. 19, 2023 (hereinafter the '399 Patent Application), which is incorporated by reference herein in its entirety.

As mentioned, in some implementations, the map acquisition system 102 compares embeddings for an observed perturbation pair to determine the ground truth similarity 212 (e.g., a phenomic similarity). For example, the map acquisition system 102 determines a similarity score (e.g., utilizing a cosine similarity or other similarity metric such as a distance metric or projection metric) of the embeddings. To illustrate, the similarity score is a numerical metric representing commonalties (or lack thereof) in bioactivity between the perturbations as expressed in their phenomic data.

Furthermore, in some implementations, the map acquisition system 102 utilizes thresholds to classify a similarity score. In some embodiments, the map acquisition system 102 classifies similarity scores for perturbation pairs utilizing a biological similar classification (e.g., for compounds and perturbations that share common bioactivity), a biological independent classification (e.g., for compounds and perturbations that have orthogonal bioactivity), or a biological dissimilar classification (e.g., for compounds and perturbations that have opposite bioactivity). For example, if the similarity score exceeds an upper threshold, the map acquisition system 102 classifies the similarity as biological similar. In some implementations, the map acquisition system 102 utilizes a different number of classifications (e.g., two classifications, such as dependent and independent).

Although the description herein often refers to single cells, it will be appreciated that the map acquisition system 102 can apply perturbations and generate embeddings for a plurality of cells (e.g., a population of cells). Thus, the map acquisition system 102 can apply a first perturbation to a plurality of cells, develop the plurality of cells, and capture a plurality of images. Moreover, the map acquisition system 102 can generate a plurality of cell representation embeddings. In some implementations, the map acquisition system 102 generates a cell representation embedding from a plurality of cells (e.g., by combining cell representations from a plurality of cells to form a cell embedding for a particular perturbation). Thus, for example, the map acquisition system 102 can generate a first cell embedding by aggregating a plurality of cell representation embeddings from a plurality of cells exposed to a first perturbation. Similarly, the map acquisition system 102 can generate a second cell representation embedding by aggregating a plurality of cell representation embeddings from a plurality of cells exposed to a second perturbation. In some implementations, the map acquisition system 102 utilizes the process described in the '989 Patent.

Additionally, FIG. 2 shows the map acquisition system 102 adding the ground truth similarity 212 to a map of biology 214. For example, the map acquisition system 102 populates the map of biology 214 with the ground truth similarity 212 for the first perturbation and the second perturbation of the perturbation pair 208.

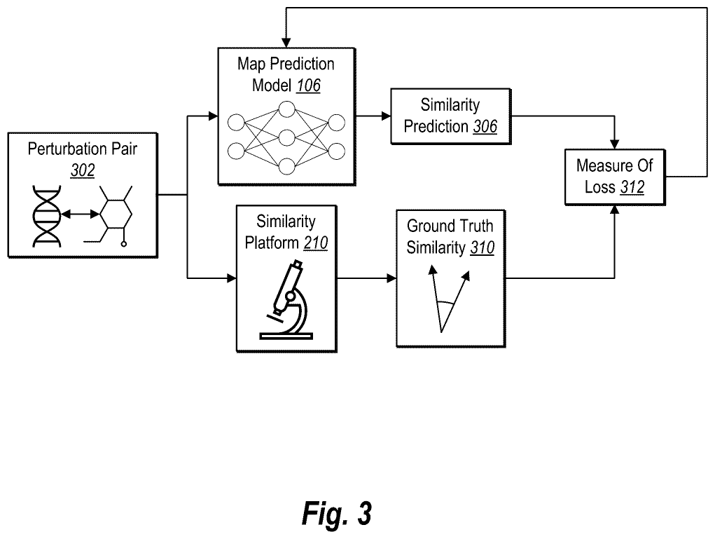

Moreover, in some embodiments, the map acquisition system 102 uses the map of biology 214 (including the ground truth similarity 212) to perform an act 216 of tuning the map prediction model 106. To illustrate, the map acquisition system 102 generates a tuned map prediction machine learning model by comparing similarity predictions for perturbation pairs with ground truth similarities. Additional detail of tuning the map prediction model 106 is given below in connection with FIG. 3.

Furthermore, FIG. 2 shows the map acquisition system 102 using a stopping criterion 218 to determine when the map prediction model 106 is satisfactorily tuned. For instance, in some embodiments, the map acquisition system 102 determines that a measure of confidence for the tuned map prediction model satisfies the stopping criterion 218. For example, the map acquisition system 102 determines that the tuned map prediction model satisfies a similarity prediction accuracy threshold (e.g., for a threshold number of tuning iterations).

To illustrate, for the stopping criterion 218, the map acquisition system 102 can utilize a 90% accuracy threshold. For example, the map acquisition system 102 can make similarity predictions on ten additional perturbation pairs, acquire ground truth results for the ten additional perturbation pairs, and then measure the accuracy of the similarity predictions against the additional perturbation pairs. If the measured accuracy (e.g., 8 of 10) fails to satisfy the accuracy threshold (e.g., 90%), the map acquisition system 102 can retrain and re-test for accuracy of the map prediction model 106. If the measured accuracy satisfies the accuracy threshold, the map acquisition system 102 can utilize the map acquisition model 106 to build a map of biology. In some implementations, utilizing the map acquisition model 106 can include retraining the map acquisition model 106 on any inconsistencies from the final set of ground truth samples, and then utilizing the retrained model without any additional assays.

As mentioned, in some implementations, the map acquisition system 102 can utilize an accuracy threshold for a threshold number of training iterations. For example, the map acquisition system 102 can utilize a stopping criterion that requires a 90% accuracy threshold over 3 training iterations. Once the map acquisition model 106 satisfies the 90% accuracy threshold for 3 training iterations, the map acquisition system 102 can utilize the map acquisition model to build a map of biology.

Moreover, in some implementations, in response to determining that the measure of confidence satisfies the stopping criterion 218, the map acquisition system 102 utilizes the tuned map prediction model to generate a blended map of biology. To illustrate, the map acquisition system 102 completes the map of biology 214 by filling in gaps with similarity predictions of the tuned map prediction model. For example, the map acquisition system 102 populates the blended map of biology with the ground truth similarity 212 for the perturbation pair 208, and generates an additional similarity prediction for an unobserved perturbation pair utilizing the tuned map prediction model.

Additional detail will now be given regarding the overall active map acquisition process. In some embodiments, the map acquisition system 102 efficiently evaluates a large, finite set of experimental designs, such as a screening compound library. The map acquisition system 102 solves a subset selection problem using an active learning strategy. Active learning is effective because, in some cases, it selects its own inference set (e.g., via inference set design). In some implementations, the map acquisition system 102 achieves high levels of performance on a target set of samples. In some embodiments, the map acquisition system 102 uses a stopping criterion to monitor the performance of the map prediction model 106 and trigger termination of the acquisition process.

To achieve efficient data acquisition of compound libraries for drug discovery applications, in some embodiments, the map acquisition system 102 seeks to reduce the number of experiments required to be run. To illustrate, the map acquisition system 102 accesses a library of compounds and a set of cell types (among other possible inputs, such as varying concentrations of the compounds and assay collection modalities) that define a target set of perturbation pairs for which to determine labels (e.g., similarity scores or classifications). For each perturbation pair, the associated experimental readout would correspond to the compound's effect on the corresponding cell type. In some implementations, the map acquisition system 102 uses binary classification labels (e.g., zero for no relationship, one for a biological similarity). Alternatively, in some implementations, the map acquisition system 102 uses continuous scores (e.g., ranging between zero and one). Moreover, in some implementations, the map acquisition system 102 uses a ternary classification (e.g., zero for no relationship, one for a biological similarity, negative one for an opposite biological relationship), or continuous scores ranging between negative one and one.

In some embodiments, the map acquisition system 102 reduces acquisition costs of screening a target set of perturbation pairs by training the map prediction model 106 on a subset of the target set, called the observation set. The map acquisition system 102 then uses the map prediction model 106 to generate predictions in place of observed labels on the remaining samples, called the inference set. As experimentation progresses, the observation set is augmented with new experimental data as additional tests are run. By contrast, the inference set diminishes over time as samples are selected from it for testing.

Upon completion of the experimental acquisition, the combination of observed labels (results for perturbation pairs in the observation set) and inferred predictions (outputs of the map prediction model 106 for perturbation pairs in the inference set) are included in a blended map of biology. Thus, a blended map of biology includes a hybrid set of labels for the entire target set—some measured and some predicted.

While acquiring all the labels via experimental observation would give the map of biology heightened accuracy, such a process would have significant expense, not only economically, but computationally. For example, the numerous experiments would be a substantial burden on computational resources, including memory requirements, storage space, network bandwidth used, and processing power (e.g., to determine embeddings for lab images and to compare the embeddings). By reducing (in some cases greatly) the required experiments, the map acquisition system 102 conserves computational resources, thereby enhancing system efficiency.

In some embodiments, the map acquisition system 102 solves an active learning problem to reduce (e.g., minimize) the number of samples in the observation set while reaching a threshold level of accuracy of similarity predictions. For example, the map acquisition system 102 sequentially selects subsets of data for experimental labelling to improve accuracy with better selection of training examples. In some implementations, the map acquisition system 102 only evaluates the inference set with the map prediction model 106. Moreover, in some embodiments, the map acquisition system 102 utilizes one or more acquisition functions that select difficult training examples for labelling, thereby making the inference-time task easier because the more difficult examples are pruned out of the inference set.

By actively selecting more challenging examples for acquisition and directing them to the observation set, in one or more embodiments the map acquisition system 102 enhances performance by actively designing the inference set such that the map prediction model 106 excels at generating predictions. Moreover, in some implementations the map acquisition system 102 uses an explicit stopping criterion to decide when enough samples are collected to meet the accuracy threshold.

As mentioned, in some implementations, the map acquisition system 102 evaluates an acquisition function over the inference set to select one or more perturbation pairs to test. To design an inference set that is easier on the map prediction model 106, the map acquisition system 102 selects perturbation pairs for experimentation that are the more difficult examples for inference. To this end, in some embodiments, the map acquisition system 102 evaluates least-confidence sampling and query-by-committee metrics. Least-confidence sampling leverages the model's class prediction probabilities as a confidence metric, selecting samples for which the map prediction model 106 exhibits the lowest predicted probabilities. The query-by-committee approach trains an ensemble of independent predictors in parallel, selecting samples where the ensemble members exhibit the highest disagreement (e.g., maximizing the entropy over the voting distribution of the ensemble members). In this way, the map acquisition system 102 identifies regions of epistemic uncertainty where model disagreement may stem from over confidence.

In some cases, a key technical challenge of inference set design is determining when to stop running new experiments. In a production system, the labels for the inference set remain unavailable. Thus, the map acquisition system 102 estimates the performance from the collected data. To avoid unnecessary experimentation, the map acquisition system 102 employs an efficient stopping criterion. To illustrate, the map acquisition system 102 maintains a probabilistic lower bound on the system accuracy, stopping when the bound exceeds the similarity prediction accuracy threshold. In some implementations, the map acquisition system 102 maintains such a bound by leveraging the feedback from each round of experimentation. As the map acquisition system 102 selects examples that the map prediction model 106 regards as challenging, the performance on batches of perturbation pairs will lower-bound the performance on the inference set.

As mentioned, in some embodiments, the map acquisition system 102 trains a map prediction model to generate similarity predictions for perturbation pairs. For instance, FIG. 3 illustrates the map acquisition system 102 tuning the map prediction model 106 based on comparisons between similarity predictions and ground truth similarities in accordance with one or more embodiments.

Specifically, FIG. 3 shows the map acquisition system 102 accessing a perturbation pair 302 and utilizing the map prediction model 106 to determine a similarity prediction 306 for the perturbation pair 302. In addition, FIG. 3 shows the map acquisition system 102 utilizing the similarity platform 210 to develop a ground truth similarity 310 for the perturbation pair 302. For example, and as described above and in additional detail below, the map acquisition system 102 utilizes the similarity platform 210 to capture phenomic images of cells exposed to the perturbations of the perturbation pair 302. Additionally, the map acquisition system 102 uses the similarity platform 210 to determine machine learning embeddings corresponding to the perturbations and to compare the embeddings to determine the ground truth similarity 310.

Moreover, in some implementations, the map acquisition system 102 compares the similarity prediction 306 with the ground truth similarity 310 to determine a measure of loss 312. The map acquisition system 102 utilizes the measure of loss 312 to tune the map prediction model 106 to predict similarities between perturbations. For example, the map acquisition system 102 adjusts parameters of the map prediction model 106 to reduce the measure of loss 312 for a subsequent tuning iteration. For instance, the map acquisition system 102 can utilize back-propagation and/or gradient descent techniques to modify parameters of the map prediction model 106 to reduce the measure of loss 312. The map acquisition system 102 can iteratively perform such training processes (e.g., utilizing different training batches) to tune the map prediction model 106 (e.g., until reaching a threshold number of training iterations or until reaching a threshold accuracy/measure of loss). Thus, in some embodiments, the map acquisition system 102 generates a tuned map prediction machine learning model by comparing the similarity prediction 306 with the ground truth similarity 310.

Upon training the map prediction model 106, the map acquisition system 102 can utilize the map prediction model 106 to analyze structural features of input compounds and generate similarity predictions for the input compounds with respect to other cell perturbations. For example, the map acquisition system 102 can predict whether a compound will be pheno-similar to, pheno-independent of, or pheno-dissimilar to one or more genes (or other perturbations, such as other chemical compounds). In some implementations, the map acquisition system 102 can generate other predictions (e.g., different classifications such as dependent or independent, or numerical similarity score predictions). Moreover, in some implementations, the map acquisition system 102 can generate confidence scores for the similarity predictions.

In some alternative embodiments, the map acquisition system 102 trains the map prediction model 106 utilizing other types of biological data than phenomic images and phenomic image embeddings. For example, in some implementations, the map acquisition system 102 utilizes RNA data applicable to a gene perturbation. For example, the map acquisition system 102 determines biosimilarity predictions for a compound or gene perturbation based on RNA counts associated with the gene perturbation. To illustrate, the map acquisition system 102 can perform perturbation assays and (instead of capturing digital images), utilize sequencing machines to determine a number of transcription proteins (e.g., mRNA) resulting from the perturbation. The map acquisition system 102 can build a transcriptomic profile for a perturbation that reflects the number of transcription proteins resulting from a particular perturbation. The map acquisition system 102 can also build a transcriptomic matrix indicating transcriptomic profiles across a plurality of perturbations. Further, the map acquisition system 102 can compare transcriptomic profiles of different perturbations to determine transcriptomic similarity scores.

In some implementations, the map acquisition system 102 generates transcriptomic embeddings based on transcriptomic profiles. For example, the map acquisition system 102 processes a transcriptomic profile utilizing a machine learning model to generate a transcriptomic embedding. In some implementations, the map acquisition system 102 can generate a transcriptomic embedding utilizing a machine learning model described in the '399 Patent Application or another embedding model. The map acquisition system 102 can compare the transcriptomic embeddings (e.g., utilizing cosine similarity or another distance measure) to generate transcriptomic similarity scores.

In some implementations, the map acquisition system 102 utilizes these transcriptomic similarity scores (rather than phenomic similarity measures) to train a machine learning model. Thus, the map acquisition system 102 can utilize a machine learning model to predict transcriptomic similarity. In addition to phenomic and transcriptomic similarity, the map acquisition system 102 can also train a machine learning model to generate other -omics predictions (e.g., inivomics predictions reflecting liability predictions or reactions of animals exposed to a particular perturbation).

Although FIG. 3 illustrates analyzing perturbation pairs in training the map prediction model 106, the map acquisition system 102 can also provide other inputs or features to the map prediction model 106 to generate similarity predictions. Indeed, the map prediction model 106 can include different input channels for analyzing different features in generating the similarity prediction 306 for different maps of biology. For example, the map acquisition system 102 can provide compound concentration features (e.g., concentration of a compound perturbation), cell type features (e.g., what cell type is used in a perturbation assay), assay modality features (e.g., the type of assay, such as cell painting or brightfield assays), and/or time series data (e.g., times of phenotype measured for a particular perturbation assay) to the map prediction model 106 in generating the similarity prediction 306. The map acquisition system 102 can thus, determine the measure of loss 312 and modify parameters of the map prediction model 106 to learn different similarity predictions based on various different input features.

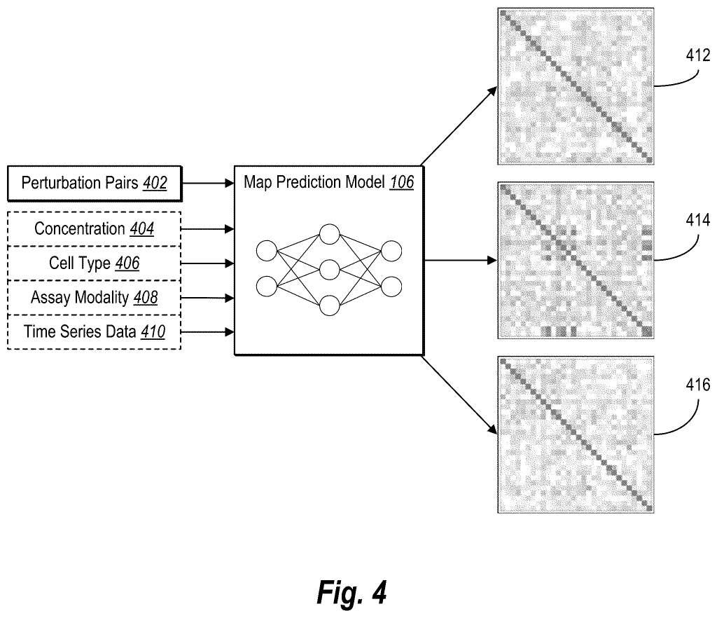

As mentioned, in some embodiments, the map acquisition system 102 generates multiple maps of biology. For instance, FIG. 4 illustrates the map acquisition system 102 generating a plurality of maps of biology based on various inputs to a map prediction model in accordance with one or more embodiments.

Specifically, FIG. 4 shows the map acquisition system 102 accessing perturbation pairs 402 for which to determine similarity predictions. Additionally, FIG. 4 shows the map acquisition system 102 using a map prediction machine learning model 420 (e.g., a tuned map prediction model, such as the map prediction model 106 when tuned) to generate a plurality of maps of biology 412-416. To illustrate, the map acquisition system 102 processes the perturbation pairs 402 through the map prediction model 420 to determine similarity predictions for the perturbation pairs 402 and populate the map of biology 412 with the similarity predictions.

As mentioned, in some embodiments, the map acquisition system 102 generates maps of biology across multiple dimensions (e.g., based on additional inputs besides chemical compounds and gene knockouts). For instance, the map acquisition system 102 utilizes compound concentration 404, cell type 406, assay modality 408, and/or time series data 410 as inputs to the map prediction model 420. Thus, the map acquisition system 102 can generate maps of biology according to additional dimensions beyond pairing cell perturbations in a matrix.

To illustrate, in some implementations, the map acquisition system 102 generates one or more maps of biology based on concentration 404 of chemical compounds. For example, the map acquisition system 102 generates a first map of biology comprising a first set of perturbation similarities at a first concentration and a second map of biology comprising a second set of perturbation similarities at a second concentration. Specifically, in training the map prediction model 106 (as described in relation to FIG. 3), the map acquisition system 102 can include concentration inputs (e.g., via a concentration channel of the map prediction model 106). The map acquisition system 102 can thus train the map prediction model 106 to generated predicted similarity scores based on an input compound concentration.

To illustrate another example, in some embodiments, the map acquisition system 102 generates one or more maps of biology based on cell type 406. For example, the map acquisition system 102 generates a first map of biology comprising a first set of perturbation similarities for a first cell type and a second map of biology comprising a second set of perturbation similarities for a second cell type. To illustrate, a mentioned above the map acquisition system 102 can include cell type as an input in training the map prediction model 106. The map acquisition system 102 can thus train and utilize the map prediction model 106 to generated predicted similarity scores based on cell type.

In some embodiments, the map acquisition system 102 performs cross-map generalization. For example, the map acquisition system 102 develops a map of biology for a given cell type at a first concentration and translates the map of biology to additional maps of biology at other concentrations. In some cases, the map acquisition system 102 generates one or more blended map of biology for one or more compound concentrations and then infers additional prediction data for unseen concentrations on the given cell type. For example, the map acquisition system 102 uses knowledge of how the additional concentration levels affect biological cell responses on other, observed cell types.

Similarly, in some embodiments, the map acquisition system 102 performs map translation across cell types. For instance, the map acquisition system 102 develops a map of biology for a first cell type (e.g., HUVEC) and then generalizes the biological similarity data across maps for other cell types (e.g., TNF alpha) with a reduced experimental burden (e.g., a smaller observation set) on those cell types.

Additionally, in some embodiments, the map acquisition system 102 generates one or more maps of biology based on assay modality 408. For example, the map acquisition system 102 generates a first map of biology comprising a first set of perturbation similarities for a first assay modality (e.g., cell painting) and a second map of biology comprising a second set of perturbation similarities for a second assay modality (e.g., bright-field microscopy). In some cases, the map acquisition system 102 generalizes results across modalities based on known patterns of biological data for different modalities. For example, the map acquisition system 102 may successfully populate a blended map of biology for bright-field data with a large inference set (and consequently a smaller observation set) based on patterns of similar data obtained from cell-painted imagery.

Moreover, in some implementations, the map acquisition system 102 generates one or more maps of biology utilizing time series data 410. For instance, the map acquisition system 102 generates a first map of biology comprising a first set of perturbation similarities at a first time lapse (e.g., two days after application of the perturbations) and a second map of biology comprising a second set of perturbation similarities at a second time lapse (e.g., ten days after application of the perturbations). For instance, the map acquisition system 102 acquires observation data via brightfield assays over a period of time. This way, the map acquisition system 102 can observe variations in biological effect of perturbations on cells over time, and generate time-series maps of biology to develop a corpus of data for understanding temporal relationships between perturbation pairs.

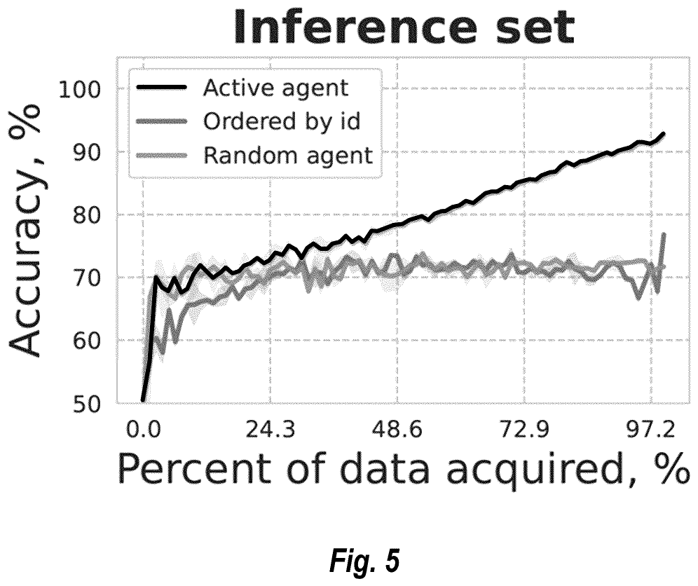

As mentioned, experiments were conducted to validate the performance of the map acquisition system 102. FIG. 5 illustrates experimental results for a pheno-similarity classification task of the map acquisition system 102 in accordance with one or more embodiments.

More particularly, the experiments conducted applied the active learning techniques described herein to a challenging real-world case of compound library screening on a large scale of phenotypic assays. Moreover, the active agent approach of the map acquisition system 102 was compared against a random agent baseline as well as a heuristic-based ordering (e.g., molecular size).

Modern high-throughput screening platforms combined with genetic perturbation techniques facilitate large scale cell microscopy experiments designed to capture the effects of biological perturbations. In some embodiments, the map acquisition system 102 performs phenotypic experimentation as follows. The map acquisition system 102 applies a perturbation to a cell before taking a microscopy image. In particular, the cells undergo either a gene perturbation (e.g., CRISPR/Cas9-mediated gene knockout) or a compound perturbation (e.g., injection of a bioactive molecular compound at a given concentration). The map acquisition system 102 processes the cell microscopy images through a neural network to obtain embeddings that correspond to a specific perturbation.

Moreover, the map acquisition system 102 constructs maps of biology from such high-throughput screening data. The maps of biology contain organized information about known and new biological relationships. A point on the map corresponds to the cosine similarity between perturbation embeddings and reveals how strongly two perturbations (i.e., a perturbation pair) are related. By expanding maps of biology, the map acquisition system 102 uncovers new biological relationships that in turn guide the discovery of leads for new medicines.

However, as discussed above, even with high-throughput screening platforms, experimentally acquiring microscopy images of all possible cell perturbations is unfeasible. In some cases, the map acquisition system 102 reduces costs associated with building maps of biology by applying active learning and inference set design to the map acquisition process. In some implementations, the map acquisition system 102 trains a map prediction model to predict the cosine similarity between perturbation embeddings of a perturbation pair. Moreover, in some embodiments, the map acquisition system 102 uses a classification approach (e.g., by classifying a prediction for a perturbation pair as biologically similar or not).

In some embodiments, the map acquisition system 102 applies a perturbation type (e.g., gene-guide pair or compound-concentration pair) with several replicates across wells, plates, and experiments. In some cases, plates also contain unperturbed control cells to track and eliminate a portion of batch effects. In some embodiments, the map acquisition system 102 aligns raw embeddings by centering and scaling them to embeddings of experiment-level unperturbed control wells. In addition, the map acquisition system 102 aggregates the embeddings through a multi-stage averaging procedure across wells, plates, experiments, and guides (for gene perturbations), yielding an average embedding for each gene-perturbation and each compound-concentration perturbation.

In some embodiments, for each gene-compound perturbation pair, the map acquisition system 102 selects the compound-concentration perturbation that yields the largest cosine similarity with the target gene. The map acquisition system 102 discretizes the cosine similarities by computing a gene-specific threshold using the upper semi-interquartile range (SIQR) with a step size of 2.5. Furthermore, the map acquisition system 102 removes genes that have activity below 25%. In some embodiments, the resulting map is a matrix of labels encoding either pheno-similarity or an absence of relationship between a gene and a compound perturbation.

In some implementations, the map acquisition system 102 treats all gene embeddings as known and focuses on the much larger space of compound acquisition. At each active learning step, the map acquisition system 102 acquires a batch of compounds from the inference set and uncovers their relationships with the genes in the dataset.

FIG. 5 shows performance of the map acquisition system 102 for a pheno-similarity classification task for a proprietary dataset. As shown in FIG. 5, the map acquisition system 102 utilizing inference set design active learning as described herein (the active agent) outperforms existing baselines. These experiments show promising results for large scale applications of active learning to hybrid screens in drug discovery programs.

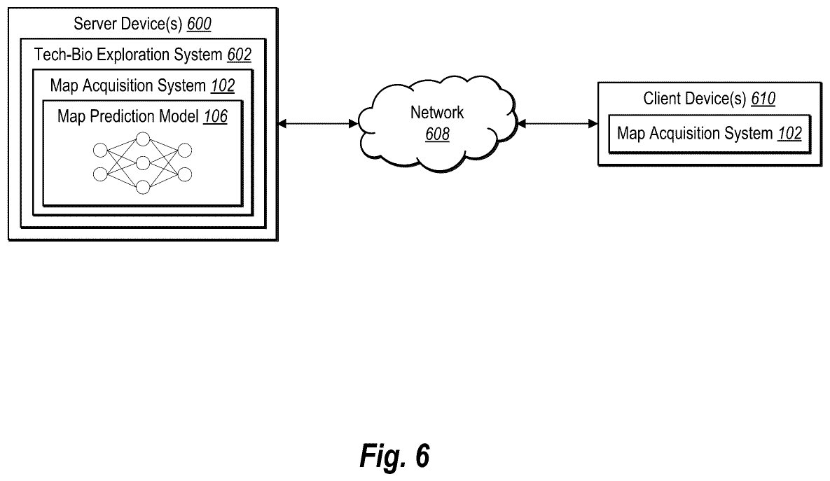

Additional detail regarding the computing environment in which the map acquisition system 102 operates will now be provided with reference to FIG. 6. In particular, FIG. 6 illustrates a schematic diagram of a system environment in which the map acquisition system 102 can operate in accordance with one or more embodiments.

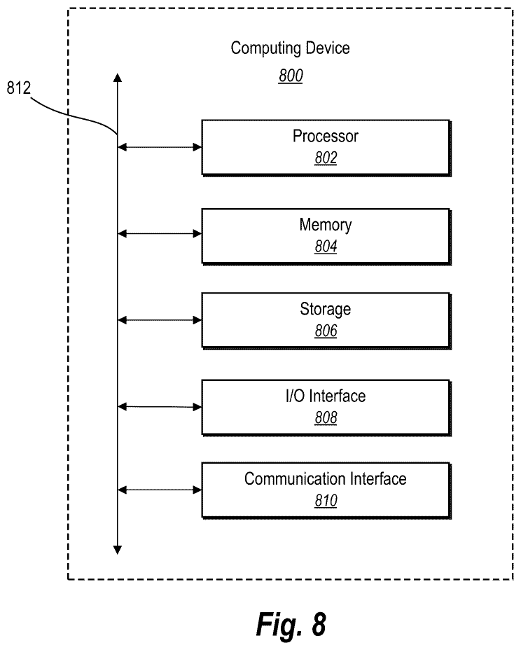

As shown in FIG. 6, the environment includes server device(s) 600 (which includes a tech-bio exploration system 602 and the map acquisition system 102), client device(s) 610, and a network 608. As further illustrated in FIG. 6, the various computing devices within the environment can communicate via the network 608. Although FIG. 6 illustrates the map acquisition system 102 being implemented by a particular component and/or device within the environment, the map acquisition system 102 can be implemented—in whole or in part—by other computing devices and/or components in the environment (e.g., additional client device(s)). Additional description regarding the illustrated computing devices is provided with respect to FIG. 8 below.

As shown in FIG. 6, the server device(s) 600 (e.g., one or more local servers operated by a particular entity) can include the tech-bio exploration system 602. In some embodiments, the tech-bio exploration system 602 can determine, store, generate, and/or provide for display tech-bio information including maps of biology, experiments from various sources, and/or machine learning tech-bio predictions. For instance, the tech-bio exploration system 602 can analyze data signals corresponding to various treatments or interventions (e.g., compounds or biologics) and the corresponding relationships in genetics, proteomics, phenomics (e.g., cellular phenotypes), and invivomics (e.g., expressions or results within a living animal). Moreover, the tech-bio exploration system 602 provides an environment for operating, executing, and/or managing complex drug discovery pipelines.

For instance, the tech-bio exploration system 602 can generate and access experimental results corresponding to gene sequences, protein shapes/folding, protein/compound interactions, phenotypes resulting from various interventions or perturbations (e.g., gene knockout sequences or compound treatments), and/or in vivo experimentation on various treatments in living animals. By analyzing these signals (e.g., utilizing various machine learning models), the tech-bio exploration system 602 can generate or determine a variety of predictions and inter-relationships for improving treatments/interventions.

To illustrate, the tech-bio exploration system 602 can generate maps of biology indicating biological inter-relationships or similarities between these various input signals to discover potential new treatments as part of the complex compound discovery process. For example, the tech-bio exploration system 602 can utilize machine learning and/or maps of biology to identify a similarity between a first gene associated with disease treatment and a second gene previously unassociated with the disease based on a similarity in resulting phenotypes from gene knockout experiments. The tech-bio exploration system 602 can then identify new treatments based on the gene similarity (e.g., by targeting compounds that impact the second gene). Similarly, the tech-bio exploration system 602 can analyze signals from a variety of sources (e.g., protein interactions, in vivo experiments) to predict efficacious treatments based on various levels of biological data.

The tech-bio exploration system 602 can generate GUIs comprising dynamic user interface elements to convey tech-bio information and receive user input for intelligently exploring tech-bio information. Indeed, the tech-bio exploration system 602 can generate GUIs displaying different maps of biology that intuitively and efficiently express complex interactions between different biological systems for identifying improved treatment solutions. Furthermore, the tech-bio exploration system 602 can also electronically communicate tech-bio information between various computing devices.

The tech-bio exploration system 602 can include a system that facilitates various models or algorithms for generating maps of biology (e.g., maps or visualizations illustrating similarities or relationships between genes, proteins, diseases, compounds, and/or treatments) and discovering new treatment options over one or more networks. For example, the tech-bio exploration system 602 collects, manages, and transmits data across a variety of different entities, accounts, and devices. In some cases, the tech-bio exploration system 602 is a network system that facilitates access to (and analysis of) tech-bio information within a centralized operating system. Indeed, the tech-bio exploration system 602 can link data from different network-based research institutions to generate and analyze maps of biology.

As shown in FIG. 6, the tech-bio exploration system 602 can include the map acquisition system 102 that generates, stores, manages, and/or transmits data pertaining to biological similarities. For example, in the context of the above description for the tech-bio exploration system 602, in some embodiments, the tech-bio exploration system 602 further utilizes the map acquisition system 102 to enhance the coordination between various groups involved in the drug discovery process. For instance, the map acquisition system 102 works in tandem with the tech-bio exploration system 602 to generate blended maps of biology, transmit the blended maps of biology to one or more devices, and initiate one or more downstream model predictions or processes.

As also illustrated in FIG. 6, the environment includes the client device(s) 610. As mentioned above, the client device(s) 610 can be involved in the process of drug discovery. Thus, for example, the client device(s) 610 can coordinate/manage a first stage of generating a blended map of biology (e.g., for a cell type with a given concentration of compounds added). Moreover, the client device(s) 610 can coordinate/manage a second stage such as generating a bioactivity prediction based on one or more similarity predictions in the blended map of biology. Further, the client device(s) 610 can coordinate and/or manage a third stage of utilizing the bioactivity prediction to generate one or more additional predictions or initiate one or more programs (e.g., industrial program generation (IPG) or industrialized compound generation (ICG)).

To illustrate, the client device(s) 610 can include computing devices that implement or manage a compound program generation stage of a compound discovery process. Similarly, the client device(s) 610 can include computing devices that implement or manage a compound lead generation stage and the client device(s) 610 can include computing devices that implement or manage a compound/dose selection stage. For example, the map acquisition system 102 can receive one or more requests to utilize the map prediction model 106 to generate one or more similarity predictions. For instance, the map acquisition system 102 can receive additional requests from the client device(s) 610 that include generating the bioactivity predictions.

In some embodiments, the environment also includes additional device(s). For example, the map acquisition system 102 can utilize the additional device(s) to further operate and manage the completion of complex drug discovery pipelines. For instance, the additional device(s) include experimental device(s) and analytical device(s). Further, in some instances, the additional device(s) also include the computing devices discussed below in connection with FIG. 8.

Furthermore, in one or more implementations, the client device(s) 610 include a client application. The client application can include instructions that (upon execution) cause the client device(s) 610 to perform various actions. For example, a user of a user account can interact with the client application on the client device(s) 610 to execute experiments or other multi-faceted processes, to further access tech-bio information, and/or initiate a request for a perturbation pair similarity prediction. For instance, in some embodiments the map acquisition system 102 receives a request to generate a similarity prediction, and in response generates the prediction and returns the prediction to the client device(s) 610. In some instances, the transmittal of the similarity prediction to the client device(s) 610 causes the client device(s) 610 to execute an action (e.g., generate a downstream model prediction or other task).

Additionally, the environment can include dedicated machine learning device(s). For example, the dedicated machine learning device(s) can include computing devices or virtual machines dedicated to training or implementing large-scale machine learning models. For example, the dedicated machine learning device(s) can generate machine learning predictions and/or embeddings based on digital biological data (e.g., digital images of phenotypes resulting from different perturbations or compound-protein interactions from compound features). Thus, the map acquisition system 102 can interact with the dedicated machine learning device(s) to generate similarity predictions.

The environment can also include experimental device(s). For example, the tech-bio exploration system 602 can interact with experimental device(s) that include intelligent robotic devices and camera devices for generating and capturing digital images of cellular phenotypes resulting from different perturbations (e.g., genetic knockouts or compound treatments of stem cells). Similarly, the experimental device(s) can include camera devices and/or other sensors (e.g., heat or motion sensors) capturing real-time information from animals as part of in vivo experimentation. The tech-bio exploration system 602 can also interact with a variety of other experimental device(s) such as devices for determining, generating, or extracting gene sequences or protein information. For example, the experimental device(s) may include computing devices linked to biosensorselectrophysiological platforms, x-ray crystallography machines, liquid chromatography mass spectrometry systems, nuclear magnetic resonance spectrometers, and/or mass spectrometers. In some implementations, the map acquisition system 102 generates similarity predictions and further determines to employ or utilize one or more experimental devices (e.g., to initiate one or more experiments based on the similarity predictions).

As further shown in FIG. 6, the environment includes the network 608. As mentioned above, the network 608 can enable communication between components of the environment. In one or more embodiments, the network 608 may include a suitable network and may communicate using a various number of communication platforms and technologies suitable for transmitting data and/or communication signals, examples of which are described with reference to FIG. 8. Furthermore, although FIG. 6 illustrates computing devices communicating via the network 608, the various components of the environment can communicate and/or interact via other methods (e.g., communicate directly).

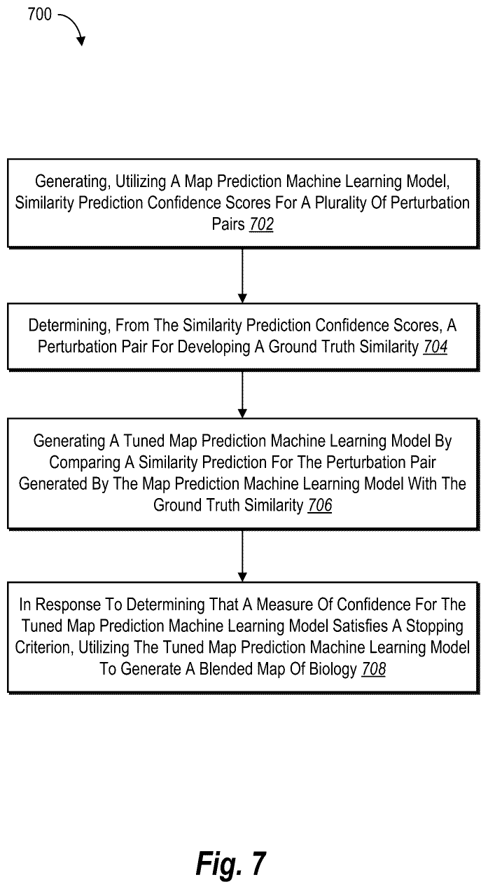

FIGS. 1-6, the corresponding text, and the examples provide a number of different methods, systems, devices, and non-transitory computer-readable media of the map acquisition system 102. In addition to the foregoing, one or more embodiments are described in terms of flowcharts comprising acts for accomplishing a particular result, as shown in FIG. 7. In some implementations, the processes of the map acquisition system 102 are performed with more or fewer acts. Furthermore, in various implementations, the acts are performed in differing orders. Additionally, in some implementations, the acts described herein are repeated or performed in parallel with one another or in parallel with different instances of the same or similar acts.

As mentioned, FIG. 7 illustrates a flowchart of a series of acts 700 for generating maps of biology via active map acquisition in accordance with one or more implementations. While FIG. 7 illustrates acts according to one implementation, alternative implementations omit, add to, reorder, and/or modify any of the acts shown in FIG. 7. In one or more implementations, the acts of FIG. 7 are performed as part of a method (e.g., a computer-implemented method). Alternatively, in one or more implementations, a non-transitory computer-readable storage medium comprises instructions that, when executed by one or more processors, cause a computing device to perform the acts of FIG. 7. In some implementations, a system performs the acts of FIG. 7.

As shown in FIG. 7, the series of acts 700 includes an act 702 of generating, utilizing a map prediction machine learning model, similarity prediction confidence scores for a plurality of perturbation pairs, an act 704 of determining, from the similarity prediction confidence scores, a perturbation pair for developing a ground truth similarity, an act 706 of generating a tuned map prediction machine learning model by comparing a similarity prediction for the perturbation pair generated by the map prediction machine learning model with the ground truth similarity, and an act 708 of utilizing, in response to determining that a measure of confidence for the tuned map prediction machine learning model satisfies a stopping criterion, the tuned map prediction machine learning model to generate a blended map of biology.

In particular, in some implementations, the act 702 includes generating, utilizing a map prediction machine learning model, similarity prediction confidence scores for a plurality of perturbation pairs, the act 704 includes determining, from the similarity prediction confidence scores utilizing an acquisition function, a perturbation pair for developing a ground truth similarity, the perturbation pair comprising a first perturbation and a second perturbation, the act 706 includes generating a tuned map prediction machine learning model by comparing a similarity prediction for the perturbation pair generated by the map prediction machine learning model with the ground truth similarity, and the act 708 includes in response to determining that a measure of confidence for the tuned map prediction machine learning model satisfies a stopping criterion, utilizing the tuned map prediction machine learning model to generate a blended map of biology.

For example, in some implementations, the series of acts 700 includes determining the perturbation pair for developing the ground truth similarity by utilizing the acquisition function to determine that a confidence value for the perturbation pair satisfies a testing criterion. Additionally, in some implementations, the series of acts 700 includes generating the ground truth similarity by comparing a first embedding for the first perturbation with a second embedding for the second perturbation. Moreover, in some implementations, the series of acts 700 includes generating the tuned map prediction machine learning model by: comparing the similarity prediction with the ground truth similarity to determine a measure of loss; and adjusting parameters of the map prediction machine learning model to reduce the measure of loss for a subsequent tuning iteration.

Furthermore, in some implementations, the series of acts 700 includes determining that the measure of confidence satisfies the stopping criterion comprises determining that the tuned map prediction machine learning model satisfies a similarity prediction accuracy threshold for a threshold number of tuning iterations. In addition, in some implementations, the series of acts 700 includes generating the blended map of biology by: populating the blended map of biology with the ground truth similarity for the perturbation pair; and generating an additional similarity prediction for an unobserved perturbation pair utilizing the tuned map prediction machine learning model.

Moreover, in some implementations, the series of acts 700 includes generating, utilizing the tuned map prediction machine learning model, a first map of biology comprising a first set of perturbation similarities for a first cell type; and generating, utilizing the tuned map prediction machine learning model, a second map of biology comprising a second set of perturbation similarities for a second cell type. Furthermore, in some implementations, the series of acts 700 includes generating, utilizing the tuned map prediction machine learning model, a first map of biology comprising a first set of perturbation similarities at a first concentration; and generating, utilizing the tuned map prediction machine learning model, a second map of biology comprising a second set of perturbation similarities at a second concentration.

Embodiments of the present disclosure may comprise or utilize a special purpose or general purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions from a non-transitory computer-readable medium (e.g., memory) and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or generators and/or other electronic devices. When information is transferred, or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface generator (e.g., a “NIC”), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed by a processor, cause a general purpose computer, special purpose computer, or special purpose processing device to perform a certain function or group of functions. In some embodiments, computer-executable instructions are executed by a general purpose computer to turn the general purpose computer into a special purpose computer implementing elements of the disclosure. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program generators may be located in both local and remote memory storage devices.

Embodiments of the present disclosure can also be implemented in cloud computing environments. As used herein, the term “cloud computing” refers to a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), a web service, Platform as a Service (“PaaS”), and Infrastructure as a Service (“IaaS”). A cloud-computing model can also be deployed using different deployment models such as private cloud, community cloud, public cloud, hybrid cloud, and so forth. In addition, as used herein, the term “cloud-computing environment” refers to an environment in which cloud computing is employed.

FIG. 8 illustrates a block diagram of an example computing device 800 that may be configured to perform one or more of the processes described above. One will appreciate that one or more computing devices, such as the computing device 800, may represent the computing devices described above (e.g., the server device(s) 600 or the client device(s) 610). In one or more embodiments, the computing device 800 may be a mobile device (e.g., a mobile telephone, a smartphone, a PDA, a tablet, a laptop, a camera, a tracker, a watch, a wearable device, etc.). In some embodiments, the computing device 800 may be a non-mobile device (e.g., a desktop computer or another type of client device). Further, the computing device 800 may be a server device that includes cloud-based processing and storage capabilities.

As shown in FIG. 8, the computing device 800 can include one or more processor(s) 802, memory 804, a storage device 806, input/output interfaces 808 (or “I/O interfaces 808”), and a communication interface 810, which may be communicatively coupled by way of a communication infrastructure (e.g., bus 812). While the computing device 800 is shown in FIG. 8, the components illustrated in FIG. 8 are not intended to be limiting. Additional or alternative components may be used in other embodiments. Furthermore, in certain embodiments, the computing device 800 includes fewer components than those shown in FIG. 8. Components of the computing device 800 shown in FIG. 8 will now be described in additional detail.

In particular embodiments, the processor(s) 802 includes hardware for executing instructions, such as those making up a computer program. As an example, and not by way of limitation, to execute instructions, the processor(s) 802 may retrieve (or fetch) the instructions from an internal register, an internal cache, memory 804, or a storage device 806 and decode and execute them.

The computing device 800 includes the memory 804, which is coupled to the processor(s) 802. The memory 804 may be used for storing data, metadata, and programs for execution by the processor(s). The memory 804 may include one or more of volatile and non-volatile memories, such as Random-Access Memory (“RAM”), Read-Only Memory (“ROM”), a solid-state disk (“SSD”), Flash, Phase Change Memory (“PCM”), or other types of data storage. The memory 804 may be internal or distributed memory.

The computing device 800 includes the storage device 806 for storing data or instructions. As an example, and not by way of limitation, the storage device 806 can include a non-transitory storage medium described above. The storage device 806 may include a hard disk drive (“HDD”), flash memory, a Universal Serial Bus (“USB”) drive or a combination these or other storage devices.

As shown, the computing device 800 includes one or more I/O interfaces 808, which are provided to allow a user to provide input to (such as user strokes), receive output from, and otherwise transfer data to and from the computing device 800. These I/O interfaces 808 may include a mouse, keypad or a keyboard, a touch screen, camera, optical scanner, network interface, modem, other known I/O devices or a combination of such I/O interfaces 808. The touch screen may be activated with a stylus or a finger.

The I/O interfaces 808 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O interfaces 808 are configured to provide graphical data to a display for presentation to a user. The graphical data may be representative of one or more graphical user interfaces and/or any other graphical content as may serve a particular implementation.

The computing device 800 can further include a communication interface 810. The communication interface 810 can include hardware, software, or both. The communication interface 810 provides one or more interfaces for communication (such as, for example, packet-based communication) between the computing device and one or more other computing devices or one or more networks. As an example, and not by way of limitation, communication interface 810 may include a network interface controller (“NIC”) or network adapter for communicating with an Ethernet or other wire-based network or a wireless NIC (“WNIC”) or wireless adapter for communicating with a wireless network, such as a WI-FI. The computing device 800 can further include the bus 812. The bus 812 can include hardware, software, or both that connects components of computing device 800 to each other.

The components of the map acquisition system 102 include software, hardware, or both. For example, the components include one or more instructions stored on a computer-readable storage medium and executable by processors of one or more computing devices, such as a client device or server device. When executed by the one or more processors, in some implementations, the computer-executable instructions of the map acquisition system 102 cause the computing device(s) to perform the methods described herein. Alternatively, in one or more implementations, the components include hardware, such as a special purpose processing device to perform a certain function or group of functions. Alternatively, in some implementations, the components of the map acquisition system 102 include a combination of computer-executable instructions and hardware.

Furthermore, the components of the map acquisition system 102 are, for example, implemented as one or more operating systems, as one or more stand-alone applications, as one or more modules of an application, as one or more plug-ins, as one or more library functions, as one or more functions callable by other applications, and/or as a cloud-computing model. Thus, in some implementations, the components are implemented as a stand-alone application, such as a desktop or mobile application. Furthermore, in various implementations, the components are implemented as one or more web-based applications hosted on a remote server. In some implementations, the components are implemented in a suite of mobile device applications or “apps.”

The use in the foregoing description and in the appended claims of the terms “first,” “second,” “third,” etc., is not necessarily to connote a specific order or number of elements. Generally, the terms “first,” “second,” “third,” etc., are used to distinguish between different elements as generic identifiers. Absent a showing that the terms “first,” “second,” “third,” etc., connote a specific order, these terms should not be understood to connote a specific order. Furthermore, absent a showing that the terms “first,” “second,” “third,” etc., connote a specific number of elements, these terms should not be understood to connote a specific number of elements. For example, a first widget may be described as having a first side and a second widget may be described as having a second side. The use of the term “second side” with respect to the second widget may be to distinguish such side of the second widget from the “first side” of the first widget, and not necessarily to connote that the second widget has two sides.

In the foregoing description, the invention has been described with reference to specific exemplary embodiments thereof. Various embodiments and aspects of the invention(s) are described with reference to details discussed herein, and the accompanying drawings illustrate the various embodiments. The description above and drawings are illustrative of the invention and are not to be construed as limiting the invention. Numerous specific details are described to provide a thorough understanding of various embodiments of the present invention.

The present invention may be embodied in other specific forms without departing from its spirit or essential characteristics. The described embodiments are to be considered in all respects only as illustrative and not restrictive. For example, the methods described herein may be performed with fewer or more steps/acts or the steps/acts may be performed in differing orders. Additionally, the steps/acts described herein may be repeated or performed in parallel with one another or in parallel with different instances of the same or similar steps/acts. The scope of the invention is, therefore, indicated by the appended claims rather than by the foregoing description. All changes that come within the meaning and range of equivalency of the claims are to be embraced within their scope.

What is claimed is:

1. A computer-implemented method comprising:
generating, utilizing a map prediction machine learning model, similarity prediction confidence scores for a plurality of perturbation pairs;
determining, from the similarity prediction confidence scores utilizing an acquisition function, a perturbation pair for developing a ground truth similarity, the perturbation pair comprising a first perturbation and a second perturbation;
generating a tuned map prediction machine learning model by comparing a similarity prediction for the perturbation pair generated by the map prediction machine learning model with the ground truth similarity; and
in response to determining that a measure of confidence for the tuned map prediction machine learning model satisfies a stopping criterion, utilizing the tuned map prediction machine learning model to generate a blended map of biology.

2. The computer-implemented method of claim 1, wherein determining the perturbation pair for developing the ground truth similarity comprises utilizing the acquisition function to determine that a confidence value for the perturbation pair satisfies a testing criterion.

3. The computer-implemented method of claim 1, further comprising generating the ground truth similarity by comparing a first embedding for the first perturbation with a second embedding for the second perturbation.

4. The computer-implemented method of claim 1, wherein generating the tuned map prediction machine learning model comprises:
comparing the similarity prediction with the ground truth similarity to determine a measure of loss; and
adjusting parameters of the map prediction machine learning model to reduce the measure of loss for a subsequent tuning iteration.

5. The computer-implemented method of claim 1, wherein determining that the measure of confidence satisfies the stopping criterion comprises determining that the tuned map prediction machine learning model satisfies a similarity prediction accuracy threshold for a threshold number of tuning iterations.

6. The computer-implemented method of claim 1, wherein generating the blended map of biology comprises:
populating the blended map of biology with the ground truth similarity for the perturbation pair; and
generating an additional similarity prediction for an unobserved perturbation pair utilizing the tuned map prediction machine learning model.

7. The computer-implemented method of claim 1, further comprising:
generating, utilizing the tuned map prediction machine learning model, a first map of biology comprising a first set of perturbation similarities for a first cell type; and
generating, utilizing the tuned map prediction machine learning model, a second map of biology comprising a second set of perturbation similarities for a second cell type.

8. The computer-implemented method of claim 1, further comprising:
generating, utilizing the tuned map prediction machine learning model, a first map of biology comprising a first set of perturbation similarities at a first concentration; and
generating, utilizing the tuned map prediction machine learning model, a second map of biology comprising a second set of perturbation similarities at a second concentration.

9. A system comprising:
at least one processor; and
at least one non-transitory computer-readable storage medium storing instructions that, when executed by the at least one processor, cause the system to:
generate, utilizing a map prediction machine learning model, similarity prediction confidence scores for a plurality of perturbation pairs;
determine, from the similarity prediction confidence scores utilizing an acquisition function, a perturbation pair for developing a ground truth similarity, the perturbation pair comprising a first perturbation and a second perturbation;
generate a tuned map prediction machine learning model by comparing a similarity prediction for the perturbation pair generated by the map prediction machine learning model with the ground truth similarity; and
in response to determining that a measure of confidence for the tuned map prediction machine learning model satisfies a stopping criterion, utilize the tuned map prediction machine learning model to generate a blended map of biology.

10. The system of claim 9, wherein the at least one non-transitory computer-readable storage medium stores additional instructions that, when executed by the at least one processor, cause the system to determine the perturbation pair for developing the ground truth similarity by utilizing the acquisition function to determine that a confidence value for the perturbation pair satisfies a testing criterion.

11. The system of claim 9, wherein the at least one non-transitory computer-readable storage medium stores additional instructions that, when executed by the at least one processor, cause the system to generate the ground truth similarity by comparing a first embedding for the first perturbation with a second embedding for the second perturbation.

12. The system of claim 9, wherein the at least one non-transitory computer-readable storage medium stores additional instructions that, when executed by the at least one processor, cause the system to determine that the measure of confidence satisfies the stopping criterion by determining that the tuned map prediction machine learning model satisfies a similarity prediction accuracy threshold for a threshold number of tuning iterations.

13. The system of claim 9, wherein the at least one non-transitory computer-readable storage medium stores additional instructions that, when executed by the at least one processor, cause the system to generate the blended map of biology by:
populating the blended map of biology with the ground truth similarity for the perturbation pair; and
generating an additional similarity prediction for an unobserved perturbation pair utilizing the tuned map prediction machine learning model.

14. The system of claim 9, wherein the at least one non-transitory computer-readable storage medium stores additional instructions that, when executed by the at least one processor, cause the system to:
generate, utilizing the tuned map prediction machine learning model, a first map of biology comprising a first set of perturbation similarities for a first cell type; and
generate, utilizing the tuned map prediction machine learning model, a second map of biology comprising a second set of perturbation similarities for a second cell type.

15. A non-transitory computer-readable medium storing instructions that, when executed by at least one processor, cause a computing device to:
generate, utilizing a map prediction machine learning model, similarity prediction confidence scores for a plurality of perturbation pairs;
determine, from the similarity prediction confidence scores utilizing an acquisition function, a perturbation pair for developing a ground truth similarity, the perturbation pair comprising a first perturbation and a second perturbation;
generate a tuned map prediction machine learning model by comparing a similarity prediction for the perturbation pair generated by the map prediction machine learning model with the ground truth similarity; and
in response to determining that a measure of confidence for the tuned map prediction machine learning model satisfies a stopping criterion, utilize the tuned map prediction machine learning model to generate a blended map of biology.

16. The non-transitory computer-readable medium of claim 15, further storing additional instructions that, when executed by the at least one processor, cause the computing device to generate the tuned map prediction machine learning model by:
comparing the similarity prediction with the ground truth similarity to determine a measure of loss; and
adjusting parameters of the map prediction machine learning model to reduce the measure of loss for a subsequent tuning iteration.

17. The non-transitory computer-readable medium of claim 15, further storing additional instructions that, when executed by the at least one processor, cause the computing device to determine that the measure of confidence satisfies the stopping criterion by determining that the tuned map prediction machine learning model satisfies a similarity prediction accuracy threshold for a threshold number of tuning iterations.

18. The non-transitory computer-readable medium of claim 15, further storing additional instructions that, when executed by the at least one processor, cause the computing device to generate the blended map of biology by:
populating the blended map of biology with the ground truth similarity for the perturbation pair; and
generating an additional similarity prediction for an unobserved perturbation pair utilizing the tuned map prediction machine learning model.

19. The non-transitory computer-readable medium of claim 15, further storing additional instructions that, when executed by the at least one processor, cause the computing device to:
generate, utilizing the tuned map prediction machine learning model, a first map of biology comprising a first set of perturbation similarities for a first cell type; and
generate, utilizing the tuned map prediction machine learning model, a second map of biology comprising a second set of perturbation similarities for a second cell type.

20. The non-transitory computer-readable medium of claim 15, further storing additional instructions that, when executed by the at least one processor, cause the computing device to:
generate, utilizing the tuned map prediction machine learning model, a first map of biology comprising a first set of perturbation similarities at a first concentration; and
generate, utilizing the tuned map prediction machine learning model, a second map of biology comprising a second set of perturbation similarities at a second concentration.
